(19) United States
(12) Patent Application Publication
(10) Pub. No.: US 2004/0054342 A1
(43) Pub. Date: Mar. 18, 2004

(54) Absorbent articles having a superabsorbent retention web

(76) Inventors: Vincent B. Newbill, Cumming, GA (US); Stanley R. Kellenberger, Appleton, WI (US); Michael J. Niemeyer, Appleton, WI (US); Lawrence Howell Sawyer, Neenah, WI (US); Ruth Ann Lachapell, Menasha, WI (US); James Hongxue Wang, Appleton, WI (US); Jennifer L. Marvin, Greenville, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/246,811

(22) Filed: Sep. 18, 2002

(51) Int. Cl. A61F 13/15; A61F 13/20

(52) U.S. Cl. 604/368

(57) ABSTRACT

Absorbent articles including an absorbent material that contains superabsorbent polymer non-adhesively attached to and restrained by a nonwoven. The material, used particularly in isolated portions of the absorbent articles of the invention, has a superabsorbent retention of at least 50% according to a stirbar retention test. Examples of such absorbent articles include pant-like garments with containment flaps and/or side panels made of the absorbent material. Other examples include swim wear garments containing the absorbent material.

### BACKGROUND OF THE INVENTION

This invention is directed to a superabsorbent retention material and to absorbent articles that include the superabsorbent retention material.

Absorbent articles such as personal care garments, medical garments, athletic garments, workwear garments, and the like, often include superabsorbent materials which are capable of absorbing a considerably large amount of liquid compared to typical absorbent materials. Because of their large absorbent capacity, superabsorbent materials are effective in reducing or preventing leakage, particularly in personal care garments. However, there are a few drawbacks associated with the use of superabsorbents.

A common complaint about products that contain superabsorbent material is gel migration. When the superabsorbent becomes saturated, the superabsorbent material tends to separate from the absorbent composite and deposit itself on the wearer of the product. The separation is due, in part, to the swelling that takes place in the superabsorbent.

Gel migration and swelling are both deterrents for the use of superabsorbent material in disposable swim wear. If swollen superabsorbent makes its way out of the product and into the pool, the superabsorbent will become trapped in the pool's filter, which could cause considerable damage to the filter system. Furthermore, the swelling alone caused by the superabsorbent material is a deterrent due to the poor fit caused by the swelling. In particular, superabsorbent materials swell significantly in low ionic strength pool water. If a superabsorbent-containing swim pant absorbs a tremendous amount of liquid, as superabsorbent materials tend to do, the fit of the pant will be very poor and the wearer will be weighed down by the pant.

Various methods are known for containing superabsorbent particles inside an absorbent product. For example, U.S. Pat. No. 5,520,673, issued May 28, 1996, describes the use of a high porosity tissue to contain the superabsorbent particles inside the product during use. As another example, U.S. Pat. No. 5,458,592, issued Oct. 17, 1995, describes the use of a thermoplastic fibrous nonwoven web to contain the superabsorbent.

U.S. Pat. No. 5,962,068, issued Oct. 5, 1999, describes a nonwoven with attached absorbent particles that has an absorbent retention greater than 60% when evaluated in the retention test described therein. The test includes swelling the absorbent polymer to saturation and rolling a roller having a diameter of 105 mm, a width of 60 mm, and a weight of 4 kg across the sample 5 times at a speed of 10 cm/s. The difference in weight is used to calculate the retention. Since this patent teaches that the viscosity of the polymerizing superabsorbent droplets arriving at the substrate is high so that absorption and impregnation is less likely, the superabsorbent retention, when evaluated in the more vigorous stirbar retention test described herein, will be very poor.

There is thus a need or desire for a superabsorbent-containing material, wherein the superabsorbent is retained within the material, swelling of the superabsorbent is constrained, yet the superabsorbent provides effective absorbency.

### SUMMARY OF THE INVENTION

In response to the discussed difficulties and problems encountered in the prior art, a new superabsorbent-containing material has been discovered.

The present invention is directed to a superabsorbent retention web and absorbent articles that include the superabsorbent retention web, particularly in isolated edge portions of the articles. These absorbent articles include personal care product applications, medical garment applications, and athletic and workwear garment applications.

The absorbent material of the invention includes superabsorbent polymer non-adhesively attached to (that is, no separate adhesion material is utilized) and restrained by a nonwoven. The absorbent material has a superabsorbent retention of at least 50% according to a stirbar retention test. The stirbar retention test is a measure of superabsorbent attachment to a web when the superabsorbent has been fully swollen. The stirbar retention test was designed to simulate the worst conditions that a superabsorbent nonwoven composite would encounter inside of a swim pant.

The absorbent material has a centrifuge retention capacity of between about 4 grams per gram and about 30 grams per gram. Because of the enhanced absorbent properties of the absorbent material, and the retention of the superabsorbent by the nonwoven, the absorbent material of the invention reduces leakage and contains the superabsorbent in a manner never before seen for use in absorbent articles.

The superabsorbent retention web retains most of the superabsorbent within the material even after reaching an equilibrium level of swelling. The material also constrains the superabsorbent from fully swelling in low-ion-containing fluids like swimming pool water due to attachment of the superabsorbent to the nonwoven. The resulting absorbent material is a web with superabsorbent attached to the web with the superabsorbent polymer surrounding or bonded to the fibers of the web. The absorbent material successfully incorporates superabsorbent polymer while preventing gel escape onto skin or into the surrounding environment. Furthermore, the constrained swelling behavior of the material prevents the article from becoming excessively large and bulky because of superabsorbent swelling.

The superabsorbent polymer can be applied to the substrate by a number of suitable non-adhesive applications, including cross-linking after printing or spraying suitable polymer solutions, and/or polymerizing and cross-linking after applying suitable monomer solutions, thereby resulting in about 25 to 95% (based on total web weight) superabsorbent. For example, the superabsorbent polymer can be in solution at a concentration between about 5% and about 30% by weight. One method, in particular, includes spraying a superabsorbent monomer particulate blend solution onto the substrate, exposing the superabsorbent solution to UV and/or other suitable radiation, and heating the irradiated solution to remove moisture, as disclosed in U.S. Pat. No. 6,417,425 issued Jul. 9, 2002, herein incorporated by reference. Another method, in particular, includes coating a nonwoven with a superabsorbent polymer containing activatable cross-linkers on one or both sides of the web and activating the cross-linkers. One specific way the superabsorbent nonwoven can be used is, for example, to cut the web into pledgets, place the pledgets upon a layer of coform, airlaid, or other suitable material, and fold the material to further contain the superabsorbent web. Another specific way the superabsorbent web can be used is to apply the superabsorbent in a central portion and either e-fold, c-fold, or z-fold the rest of the web over or around the superabsorbent-treated central portion.

The nonwoven to which the superabsorbent polymer is attached can be meltspun, meltblown, coform, a meltspun laminate with, for example, barrier and/or elastomeric characteristics, bonded carded web, high loft bonded carded web, or elastomeric high loft bonded carded web, or any other suitable nonwoven.

The absorbent articles of the invention can be personal care garments, medical garments, athletic or workwear garments, or any other type of absorbent article. In particular, the absorbent article can be a pant-like absorbent garment, such as a diaper, training pants, absorbent underpants, or adult incontinence product, or absorbent swim wear, or any other absorbent products, such as sweat absorbent garments, etc. The absorbent material of the invention can also be used as a wetness indicator, or wetness awareness aid, such as for use in toilet training.

The absorbent article of the invention is particularly suitable as swim wear because the absorbent material within the absorbent article can reduce pre-swim leakage and improve a wearer's comfort, while preventing gel blockage, or filter contamination, from occurring in swimming pools. Also, the constrained swelling of the absorbent material is particularly useful in swim wear applications since superabsorbent polymer, when unconstrained, swells significantly more in low ionic strength pool water than when absorbing body fluids which contain ions.

The absorbent article of the invention is also particularly suitable as absorbent articles worn overnight or garments worn by very active wearers. By providing a way to add superabsorbent to nonwoven substrates, the resulting absorbent material can be incorporated into an absorbent article in a number of strategic locations to reduce or prevent leakage. For example, the absorbent material can be attached to the side panels of a pant-like garment or the ear portion of a diaper to reduce leakage from the side of the garment while the wearer is sleeping, especially when sleeping on their side. The side panels incorporating the absorbent material are particularly useful if a failure in the barrier flap gasketing occurs. Another strategic location of the absorbent material is in containment flaps. By providing superabsorbent polymer in containment flaps, absorbent capacity would be provided in a location that normally would not have any absorbent capacity. Therefore, if fluid ran to the containment flaps, the fluid would be absorbed by the superabsorbent on the flaps, thereby significantly reducing leakage from the product. Furthermore, this invention allows more absorbent capacity to be added to the product without adding more material to the absorbent core, thus maintaining or decreasing thickness, etc. As an additional benefit, increased product capacity without adding new components and/or elements is made possible.

With the foregoing in mind, it is a feature and advantage of the invention to provide an absorbent material having considerable superabsorbent containment, effective absorbency, and the ability to remain relatively thin when in a fully swollen state.

### BRIEF DESCRIPTION OF THE DRAWINGS

FIGS. 1a, 1b, and 1c illustrate absorbent material of the invention.

FIGS. 2a, 2b, and 2c illustrate fibers of the absorbent material of the invention.

FIG. 3a illustrates an e-folded absorbent material of the invention.

FIG. 3b illustrates a c-folded absorbent material of the invention.

FIG. 3c illustrates a z-folded absorbent material of the invention.

FIG. 4 is a perspective view of a pant-like absorbent garment, such as a swim pant, including the absorbent material of the invention.

FIG. 5 is a plan view of a pant-like absorbent garment in a partially disassembled, stretched flat state, and showing the surface of the garment that faces the wearer when the garment is worn, and with portions cut away to show the underlying features.

FIG. 6 illustrates one pattern for printing superabsorbents.

### DEFINITIONS

Within the context of this specification, each term or phrase below will include the following meaning or meanings.

“Absorbent article” includes personal care garments, medical garments, athletic and workwear garments, and the like. The term “disposable garment” includes garments which are typically disposed of after 1-5 uses. The term “personal care garment” includes diapers, training pants, swim wear, absorbent underpants, adult incontinence products, feminine hygiene products, nursing pads, underarm pads, wipes, breathable-when-dry outer absorbent product covers, and the like. The term “medical garment” includes medical (i.e., protective and/or surgical) gowns, caps, gloves, drapes, face masks, bandages, and the like. The term “athletic garments” includes athletic socks, pants, supporters, bras, shirts, sweat bands, helmet liners, and the like. The term “workwear garments” includes laboratory coats, cover-alls, hard-hat liners, and the like.

“Airlaid” refers to a material produced by forming previously individualized fiber with or without other materials and bonding them together with adhesives, glues, and/or heat-activated binder fiber.

“Attached” refers to the joining, adhering, connecting, bonding, or the like, of at least two elements. Two elements will be considered to be attached together when they are attached directly to one another or indirectly to one another, such as when each is directly attached to intermediate elements.

“Bonded carded web” refers to webs made from staple length fibers that are carded into a web and then bonded by some technique such as thermal or adhesive bonding.

“Central portion” refers to a portion of an article that is a distance away from each of the edges of the article and does not include outermost edges of the article.

“Coform” refers to a material produced by combining separate polymer and additive streams (e.g. fluff pulp) into a single deposition stream in forming a nonwoven web. Such a process is taught, for example, by U.S. Pat. No. 4,100,324 to Anderson et al. which is hereby incorporated by reference.

“Elastomeric” is the property of a material that refers to its ability to extend when under a load and recover a significant portion of the load-induced extension after the load is removed. “Elastomeric” and “elastic” are used interchangeably to refer to a material or composite that is generally capable of recovering its shape after deformation when the deforming force is removed. Specifically, as used herein, elastic or elastomeric is meant to be that property of any material which, upon application of an elongating force, permits the material to be stretchable to a stretched length which is at least about 25 percent greater than its relaxed unstretched length, and that will cause the material to recover at least 40 percent of its elongation upon release of the stretching force. A hypothetical example which would satisfy this definition of an elastomeric material would be a ten (10) centimeter sample of a material which is elongatable to at least 12.5 centimeters and which, upon being elongated to 12.5 centimeters and released, will recover to a length of less than 11.5 centimeters. Many elastic materials may be stretched by much more than 25 percent of their relaxed length, and many of these will recover to substantially their original relaxed length upon release of the stretching force.

“Elastomeric high-loft bonded carded webs” are low-density webs that contain a means of rendering them elastomeric. An example of a suitable material of this type includes two layers of through-air-bonded 17 grams per square meter (gsm) polyester with a polyethylene/polypropylene binder fiber carded web laminated to both sides of a plurality of stretched, extruded and cooled KRATON 6631 elastomeric polymer filaments, with 1.5 gsm Findley 2096 adhesive hot melt, available from Ato-Findley, Inc., Wauwatosa, Wis., sprayed on each layer of web. KRATON 6631 elastomeric polymer is available from Kraton Polymers, Belpre, Ohio.

“Encase” refers to the act of surrounding, coating, or otherwise covering an object. The term “encase” includes both total encasing and partial encasing.

“Foam” refers to two-phase gas-solid systems that have a supporting solid lattice of cell walls that are continuous throughout the structure. The gas, typically air, phase in a foam is usually distributed in void pockets often called cells. As used herein, “foam” is in the class of nonwovens.

“High-loft bonded carded webs” are low-density bonded carded webs often used for surge/acquisition functions in personal care garments.

“Isolated edge portion” refers to a portion of an article that includes at least one edge of the article but includes less than half of the surface area of the article. An article can have more than four edges, such as, for example, an article having flaps attached interior to at least one edge of the article.

“Layer” when used in the singular can have the dual meaning of a single element or a plurality of elements.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface.

“Meltspun fiber” refers to small diameter fibers which are formed by extruding molten thermoplastic material as filaments from a plurality of fine capillaries of a spinnerette having a circular or other configuration, with the diameter of the extruded filaments then being rapidly reduced as taught, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Meltspun fibers are quenched and generally not tacky when they are deposited onto a collecting surface. Meltspun fibers are generally continuous and often have average deniers larger than about 0.3, more particularly, between about 0.6 and 10. The term “spunbond” is often used synonymously with the term “meltspun,” especially when referring to a bonded web of meltspun fibers.

“Nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, meltspinning processes, air laying processes, and bonded carded web processes. The term “nonwoven,” in the most general sense, refers to any structure which is not woven and thus also includes such structures as foams.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Superabsorbent,” “superabsorbent polymer,” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as polyphosphazenes, or organic compounds such as cross-linked polymers. Also included are materials which do not swell but have high enough internal capacities such as aerogels which are capable of absorbing at least about 15 and more desirably at least about 30 times their weight in an aqueous solution containing 0.9 weight percent sodium chloride.

“Superabsorbent retention” refers to the amount of superabsorbent that remains attached to a web during or after use.

“Surge material” refers to a layer of material designed to rapidly accept fluid exudates and distribute the fluid exudates to a retention structure. Examples of suitable surge materials are described in U.S. Pat. No. 5,486,166 to Bishop and U.S. Pat. No. 5,490,846 to Ellis, both of which are hereby incorporated by reference.

These terms may be defined with additional language in the remaining portions of the specification.

### DETAILED DESCRIPTION OF THE PRESENTLY PREFERRED EMBODIMENTS

The present invention is directed to an absorbent material having considerable superabsorbent containment, effective absorbency, and the ability to remain thin when in a fully swollen state. The material can be incorporated into any suitable absorbent article, such as personal care garments, medical garments, and athletic and workwear garments. More particularly, the material is suitable for use in diapers, training pants, swim wear, absorbent underpants, adult incontinence products, feminine hygiene products, nursing pads, underarm pads, wipes, breathable-when-dry outer absorbent product covers, protective medical gowns, surgical medical gowns, bandages, caps, gloves, drapes, face masks, laboratory coats, coveralls, sweatbands, table cloths, and paper tissue, etc.

The absorbent material of the invention includes a nonwoven with a superabsorbent polymer attached to and restrained by the nonwoven without the use of bonding agents such as adhesives. The method by which the superabsorbent polymer is applied to the nonwoven is important because the method affects the properties of the resulting material.

The nonwoven included in the material of the invention may be formed using conventional processes, including the meltspun and meltblowing processes described in the DEFINITIONS. For example, the nonwoven may be a meltspun web (e.g. spunbond) having a basis weight of about 3 to 150 grams per square meter (gsm), suitably 6 to 75 gsm, or about 10 to 25 gsm. Other suitable nonwovens include coform, elastomeric high-loft bonded carded webs, meltspun laminate, surge, and high loft surge.

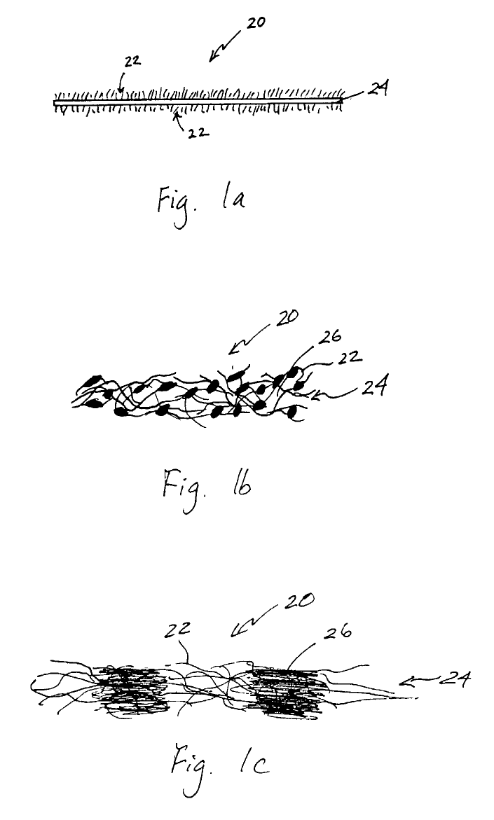

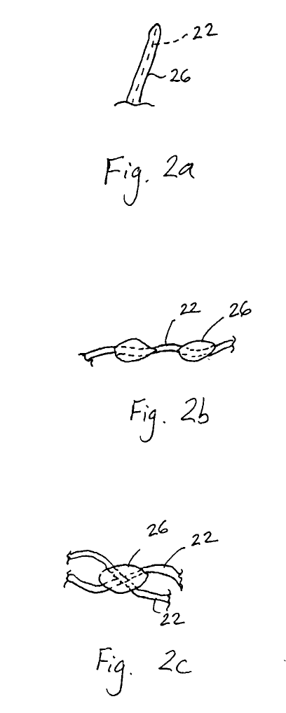

FIG. 1a illustrates a side view of the absorbent material 20 of the invention, showing fibers 22 extending from the nonwoven 24. FIG. 2a illustrates a single fiber 22, representative of many fibers, of the absorbent material 20 with a superabsorbent polymer 26 non-adhesively attached to, or encasing, the fiber 22. FIGS. 1b, 1c, 2b, and 2c illustrate alternative types of structures. In FIGS. 1b and 2b the superabsorbent 26 only partially encases the fibers 22, while in FIGS. 1c and 2c the superabsorbent 26 encases many fibers 22.

The superabsorbent polymer 26 can include, for example, alkali metal salts of polyacrylic acids; polyacrylamides; polyvinyl alcohol; ethylene maleic anhydride copolymers; polyvinyl ethers; hydroxypropylcellulose; polyvinyl morpholinone; polymers and copolymers of vinyl sulfonic acid, polyacrylates, polyacrylamides, polyvinyl pyridine; and the like. Other suitable polymers include hydrolyzed acrylonitrile grafted starch, acrylic acid grafted starch, and isobutylene maleic anhydride copolymers and mixtures thereof Further suitable polymers include inorganic polymers such as polyphosphazene and the like.

Typically, a superabsorbent material is capable of absorbing at least about 15 times its weight in 0.9 weight percent sodium chloride, and desirably is capable of absorbing more than about 30 times its weight in 0.9 weight percent sodium chloride. Suitable superabsorbent materials are available by, for example, following the teachings in U.S. Pat. No. 4,500,351 issued Feb. 19, 1985 to Peniak et al., using ISOBAM 18 available from the Kuraray America, Inc. of New York, New York, and diethylene triamine cross-linker, or the emulsion method of PCT Publication No. WO 00/50096 published Aug. 31, 2000 by Gartner et al., or using a suitable mixture of monomer, cross-linker, and initiators per the teachings in U.S. Pat. No. 6,417,425 to Whitmore et al., or the method of U.S. Pat. No. 5,962,068 issued Oct. 5, 1999 wherein the redox initiated polymerizing superabsorbent is applied to the web early enough to surround fibers, not just to have a few particles attach to the fibers, thereby leaving the rest of the particles to attach to other particles. Each of these references relating to superabsorbent materials is hereby incorporated by reference.

The superabsorbent polymer 26 is applied to the nonwoven 24 by surrounding fibers 22 in the nonwoven 24 or by bonding the superabsorbent 26 to itself or the nonwoven with crosslinkers in a superabsorbent polymer or prepolymer solution. Crosslinking may, for example, be by bonds which range from highly ionic to highly covalent types of bond, or the like. These bonds can be further augmented with hydrogen bonds and/or induced polar bonds. The superabsorbent polymer can be in a solution at a concentration of between about 5% and about 30% by weight, or between about 10% and about 25% by weight, or between about 15% and about 22% by weight. Suitably, the superabsorbent polymer is in a solution with a shear rate viscosity of between about 3,000 and about 20,000 centipoise, or between about 5,000 and about 15,000 centipoise, or between about 7,000 and about 13,000 centipoise at a shear rate of 0.1 1/s and a temperature of 22 degrees Celsius. Alternatively, the superabsorbent polymer can be formed with a suitable mixture of monomer, cross-linkers, and initiators in-situ on the web.

Methods of applying the superabsorbent polymer to the nonwoven include saturation, printing, coating, and spraying. In one particular method, namely an in-situ polymerization superabsorbent coating process, a superabsorbent monomer solution containing monomer, crosslinkers, and initiators is sprayed onto the nonwoven, the sprayed nonwoven is exposed to UV radiation and/or other radiation in order to polymerize and crosslink the monomer, and the irradiated substrate is then exposed to heat to remove any remaining moisture. In another method, two superabsorbent precursor solutions, each containing one part of a redox pair, are combined to initiate polymerization and are applied to the nonwoven before polymerization is completed to firmly attach the superabsorbent to the nonwoven. In yet another method, the nonwoven is coated, with complete coverage or only in discreet areas, on one or both sides, with superabsorbent polymer containing activatable cross-linkers which are activated to cross-link the superabsorbent polymer. The methods result in 25 to 95 percent superabsorbent polymer in the nonwoven. The nonwoven can then, for example, be cut into pledgets and placed in the center of a piece of absorbent web, such as 90 gsm coform. The coated nonwoven can then be folded inside of the coform material, thus providing additional capacity to the absorbent core of the product.

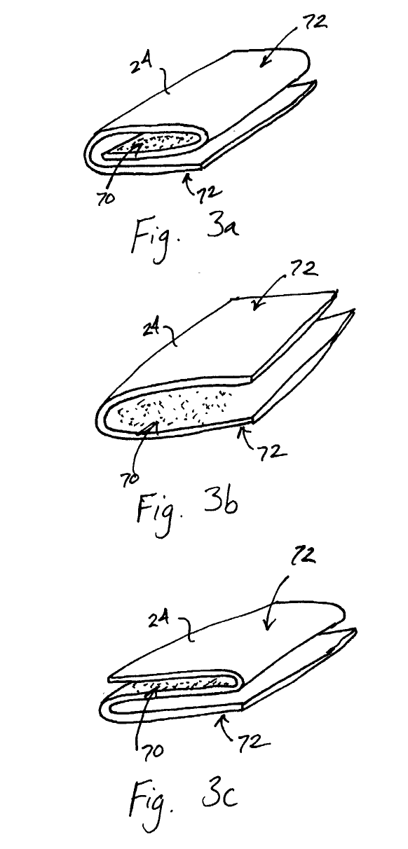

In one embodiment, the nonwoven 24 can be coated in just a discreet zone and the uncoated areas 72 can be e-folded, c-folded, or z-folded around the coated portion 70 such that the coated portion is encompassed within the folded nonwoven, and thus the coated portion 70 does not have any exposure outside the folded nonwoven. An example of an e-folded nonwoven is illustrated in FIG. 3a. An example of a c-folded nonwoven is illustrated in FIG. 3b. An example of a z-folded nonwoven is illustrated in FIG. 3c.

The resulting superabsorbent is not in the form of particles trapped by surrounding fibers but instead is in the form of particles that encase or bond firmly to fibers because the particles were polymerized or crosslinked while in contact with or even surrounding the fibers, as shown in FIGS. 2a-2c. Therefore, the superabsorbent material in this invention is not held onto the web by any type of adhesive, binder, or glue.

The resulting absorbent material has enhanced superabsorbent retention because the superabsorbent is non-adhesively but firmly adhered to the nonwoven. For instance, the nonwoven retains most of the superabsorbent within the material even after reaching an equilibrium level of swelling in water, thus preventing gel escape onto skin or into the surrounding environment. The nonwoven material also constrains the superabsorbent from fully swelling in water due to firm attachment of the superabsorbent to the nonwoven. The thickness increase, measured at 0.2 psi, after freely swelling in tap water containing at least about 7 ppm sodium, 20 ppm calcium, 6 ppm magnesium, and 0.1 ppm iron for one hour is less than about 150 percent, or less than about 100 percent, or less than about 75 percent, or less than about 50 percent more than the thickness increase when swelling for one hour in 0.9 weight percent saline.

The absorbent material provides improved absorbent capacity of nonwovens with the fluid lockup capability of the superabsorbent. More particularly, the material has a centrifuge retention capacity that ranges from about 4 to at least about 30 grams per gram (g/g), or from about 5 g/g to about 25 g/g, or from about 6 g/g to about 20 g/g, as measured by the EDANA 441.1-99 Centrifuge Retention Capacity test wherein the complete nonwoven with superabsorbent is evaluated, and is highly dependent upon the technology used to apply the superabsorbent to the nonwoven and the properties of the superabsorbent polymer. This is a large improvement in centrifuge capacity over the original capacity of nonwovens which is typically less than 2.5 g/g.

A stirbar retention test, described in detail below, was used to evaluate superabsorbent retention. The test measures superabsorbent attachment to a web when the superabsorbent has been fully swollen. The test was designed to simulate the worst conditions that a superabsorbent nonwoven composite would encounter inside of a swim pant. The superabsorbent retention of the material of the invention is at least 50%, or at least 60%, or at least 70%, or at least 80%, according to the stirbar retention test.

Disposable swimwear with absorbent material of the invention, when subjected to a forced failure test, described in detail below, resulted in a significant reduction in the probability of pant failure. The forced failure test evaluates the maximum load of 0.9 weight percent sodium chloride that the test sample will absorb before the sample leaks. The results are reported in terms of the load on the sample when leakage first occurs. For the absorbent material of the invention, the average load at leak was increased by 22 grams in one case and 40 grams in the example below, according to the forced failure test.

The absorbent material 20 of the invention is particularly suitable for use in absorbent articles, especially in isolated edge portions of absorbent articles. In one embodiment, the absorbent material 20 may be used along one or more isolated edge portions of an absorbent article while superabsorbent polymer non-adhesively attached to a nonwoven may be present in a central portion of the article. In another embodiment, the absorbent material 20 in the isolated edge portion(s) of the absorbent article may surround non-adhesively attached absorbent material in the central portion of the article.

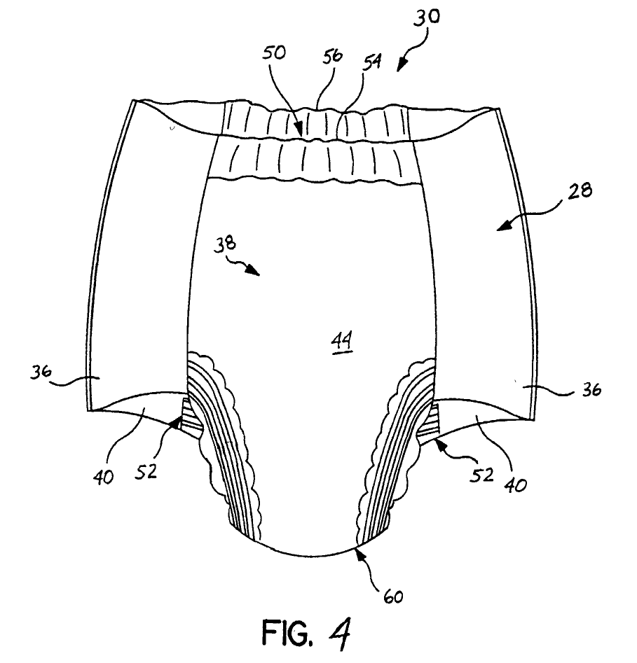

In one embodiment, the absorbent material 20 can be integrated into an absorbent chassis 28 to create a pant-like absorbent garment 30, as shown in FIG. 4. More particularly, the absorbent material 20 provides a way to add superabsorbent to nonwoven substrates and place them into a garment in regions of the garment that normally lack absorbent capacity.

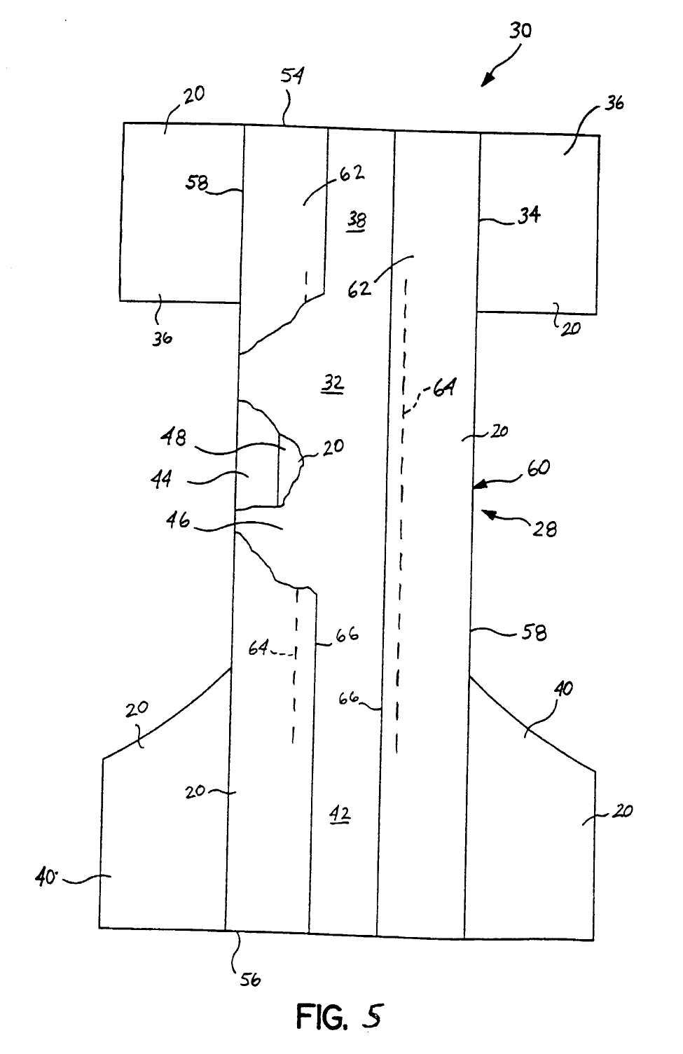

Referring to FIG. 5, the absorbent garment 30 is shown in a partially disassembled, stretched flat state, showing an inner surface 32 which faces the wearer when the garment is worn. The chassis 28 includes a somewhat rectangular composite structure 34, a pair of transversely opposed front side panels 36 extending from a front panel 38 of the composite structure, and a pair of transversely opposed back side panels 40 extending from a back panel 42 of the composite structure. The composite structure 34 and side panels 36, 40 may be integrally formed, or may include two or more separate elements, as shown in FIG. 5. Also, the front and back side panels 36, 40 may be permanently attached to one another, releasably attached to one another, or may be integrally formed with one another on either side of the garment 30.

The illustrated absorbent chassis 28 includes an outer cover 44, a body side liner 46 which is connected to the outer cover 44 in a superposed relation, and an absorbent layer 48 located between the outer cover 44 and the body side liner 46.

As shown in the garment 30 in FIG. 4, the absorbent chassis 28 defines a three-dimensional pant configuration having a waist opening 50 and a pair of leg openings 52. Front and back waist edges 54, 56 of the absorbent chassis 28 are configured to encircle the waist of the wearer when worn and provide the waist opening 50 which defines a waist perimeter dimension. Portions of transversely opposed side edges 58 (FIG. 5) in a crotch region 60 generally define the leg openings 52. The crotch region 60 of the garment 30 is situated between the front panel 38 and the back panel 42 and includes the portion of the garment 30 which, when worn, is positioned between the legs of the wearer and covers the lower torso of the wearer.

The absorbent layer 48, positioned between the outer cover 44 and the body side liner 46, is generally conformable, non-irritating to the child's skin, and capable of absorbing and retaining liquids and certain body wastes. The absorbent layer 48 can be manufactured in a wide variety of sizes and shapes.

The absorbent chassis 28 desirably, although not necessarily, includes a pair of containment flaps 62 which are configured to provide a barrier to the transverse flow of body exudates. A flap elastic member 64 (FIG. 5) is operatively joined with each containment flap 62 in any suitable manner as is well known in the art. The elasticized containment flaps 62 each define an unattached edge 66 which assumes an upright, generally perpendicular configuration in at least the crotch region 60 of the garment 30 to form a seal against the wearer's body. The containment flaps 62 can be located along the transversely opposed side edges 58 of the absorbent chassis 28, and can extend longitudinally along the entire length of the absorbent chassis or may only extend partially along the length of the absorbent chassis. Suitable constructions and arrangements for the containment flaps 62 are generally well known to those skilled in the art and are described in U.S. Pat. No. 4,704,116 issued Nov. 3, 1987 to Enloe, which is incorporated herein by reference.

The absorbent material 20 of the invention is particularly suitable for use in forming the containment flaps 62. The superabsorbent on the containment flaps 62 provides absorbent capacity to a region of the garment 30 that normally would not have any absorbent capacity. Unlike other superabsorbent applications that tend to leave gel on a wearer's skin, the absorbent material 30 of the invention is designed to prevent gel migration. Containment flaps 62 made of the absorbent material 30 are particularly advantageous in garments worn by very active wearers. If fluid runs to the flaps 62, the fluid will be absorbed by the superabsorbent on the flaps, thereby significantly reducing leakage from the garment 30.

Another suitable use for the absorbent material 20 of the invention is in the side panels 36, 40 of a diaper or other pant-like garment 30. In this embodiment, as in the previous embodiment, the superabsorbent on the side panels 36, 40 provides absorbent capacity to a region of the garment 30 that normally would not have any absorbent capacity. Side panels 36, 40 made of the absorbent material 20 are particularly advantageous in garments that are worn overnight, such as diapers and training pants. Should a failure in the barrier flap gasketing occur, the absorbent side panels 36, 40 would significantly reduce leakage from the side of the garment 30, such as when the wearer is sleeping.

The absorbent material 20 of the invention is also suitable for use in absorbent swim wear to reduce pre-swim leakage and to improve the level of comfort to the wearer. For example, the absorbent layer 48 of the swim wear garment 30 can include the absorbent material 20. The constrained swelling behavior of the absorbent material 20 is especially useful in applications like swim wear since superabsorbent polymers swell significantly more in low ionic strength pool water compared to other in-use environments. Thus, the constrained swelling behavior prevents the garment 30 from becoming excessively large and bulky because of superabsorbent swelling. Because the absorbent material 20 retains most of the superabsorbent even after reaching an equilibrium level of swelling, use of the absorbent material in swim wear can prevent gel blockage or filter contamination problems from occurring in swimming pools.

In addition to swim wear, the absorbent material 20 can be used in virtually any product to prevent gel on skin issues and/or gel escape into the surrounding environment. For example, the absorbent material 20 can be used as a wetness indicator, or wetness awareness aid, such as for use in toilet training. For instance, a piece of the absorbent material could be placed outside a bodyside liner, or may coat a portion of the bodyside liner, of a training pant or other absorbent garment without creating gel on skin problems. When the material becomes wet and swells, the wet gel will remain against the user's skin, creating a tactile sensation, such as the swollen gel or an evaporative cooling sensation that alerts the user to the fact that the absorbent garment has experienced a liquid insult.

### EXAMPLES

### Example 1

A specific printed example of the material 20 was produced using an aqueous solution of ISOBAM 18 and diethylenetriamine crosslinker printed onto an elastomeric high-loft bonded carded web and then cured as taught in co-pending U.S. patent application Ser. No. 10/036,746, filed Dec. 21, 2001, by Wang et al. Before printing with the superabsorbent solution, the coform material had a centrifuge retention capacity as taught in EDANA 441.1-99 and run at 300 G of about 2.6 g/g. Once superabsorbent was printed on the substrate at a loading of 40 weight percent (based on weight of coform and superabsorbent), the centrifuge retention capacity was about 8 g/g. This was an increase in capacity of more than about 200%.

The ISOBAM 18 solution used for the printing contained 20% solids and was 55% neutralized. It was generally made using the following method.

1. Mix 200 grams of ISOBAM 18 powder and 650 grams of distilled water in a laboratory reactor while heating at 80° C.

2. In a separate container, mix 56.8 grams of sodium hydroxide with 350 grams of distilled water.

3. After both solutions are well mixed, add the caustic solution dropwise to the polymer solution while continuing mixing and heating in the reactor.

4. Continue stirring and heating until the solution becomes clear.

5. Add 8 grams of diethylenetriamine to the clear solution and continue mixing and heating for 30 minutes.

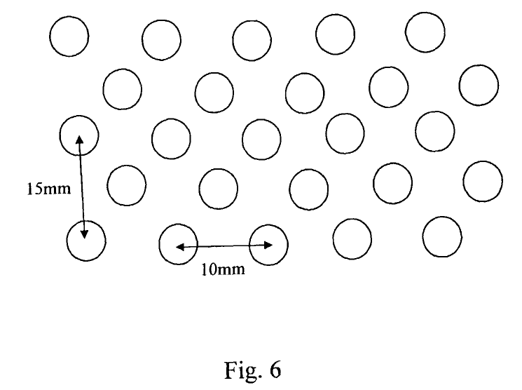

Once the aqueous solution was made, having a viscosity as determined by the method described in Example 2 of about 10,500 centipoise when measured at a low shear rate of 0.1 1/s at a temperature of 22 degrees Celsius so as to facilitate getting it into the nonwoven structure while still maintaining pattern definition, it was printed onto the substrate. In order to control the superabsorbent location and the add-on amount, a pattern was created using an approximately 1 mm thick TEFLON sheet. Approximately 4 mm diameter holes were die cut into the sheet spaced about 15 mm apart (center to center) in the length direction and about 10 mm apart (center to center) in the width direction such that they formed an off-set staggered pattern, as shown in FIG. 6. The pattern was further divided into 3 segments about 70 mm wide divided by two 10 mm wide segments that did not contain any holes. About 90 gsm (65% wood pulp fluff, 35% polypropylene meltblown) coform substrate was placed under the TEFLON sheet and superabsorbent solution was forced through the holes using a 1-inch paint brush. The solution was brushed through the holes into the material until the underside of the substrate in the areas of the holes became saturated with superabsorbent solution. The substrate was then placed in an oven for 2 hours at a temperature of 120° C. in order to dry and crosslink the superabsorbent. Afterward the superabsorbent spots were about 5 mm in diameter. This material was then tri-folded along the 10 mm spaces not containing any superabsorbent to form a structure about 80 mm wide by 330 mm long. This structure was placed in disposable swimwear in place of control tri-folded 130 gsm coform not containing superabsorbent. This material had a superabsorbent retention of greater than 70% when tested by the Stirbar Retention Test.

The absorbent tri-folded core in the material of this example was only 70% thicker when exposed to tap water (about 7 ppm sodium, 20 ppm calcium, 6 ppm magnesium, and 0.1 ppm iron) for one hour than when exposed to the urine simulant, 0.9 weight percent sodium chloride. The thickness was measured after blotting off the excess liquid with 4 layers of paper toweling 1 minute on each side using fresh paper toweling on each side and measuring the thickness at 0.2 psi taking the reading after the rate of thickness decrease is less than about 0.01 mm/15 seconds.

### Example 2

ISOBAM-18 is poly(isobutylene-co-maleic anhydride) having a molecular weight of about 300,000 to 350,000 g/mole. The more detailed procedure for making the printable superabsorbent polymer solution in Example 1 (Sample 1) is provided as follows: In a 2-liter PYREX glass resin kettle reactor (5.25 inches diameter and 7 inches in height), 200 grams of ISOBAM-18 powder and 650 grams of de-ionized water were added. The reactor was equipped with an overhead motor driven blade stirrer, a thermocouple for measuring temperatures, and a liquid addition funnel. The mixture was heated by a heating mantle which was controlled by a DYNA SENSE controller, Model 2157, supplied by Cole-Parmer Instrument Company, Chicago, Ill. The resulting mixture was stirred by a heavy-duty laboratory motor Type 6T-10, 115 volts DC, 0.6 amp, 1/20 HP, manufactured by G. K. Heller Corp., Floral Park, N.Y. The motor was controlled by a Series S motor controller manufactured by G. K. Heller Corp. at a setting of 7 or higher. The temperature was set to 80 degrees Celsius. A white slurry resulted from the mixture.

For Sample 1, a solution of sodium hydroxide was obtained by adding 54.2 grams of reagent grade sodium hydroxide, from Aldrich, and 350 grams of de-ionized water into a 500 ml beaker. The mixture was stirred by a magnetic stirring bar on a stirring plate.

When the temperature of mixture in the resin kettle reached the set temperature (80 degrees Celsius), the sodium hydroxide solution was added to the mixture dropwise through the liquid addition funnel. The mixture was again stirred at the set temperature for 4 hours. A translucent solution resulted. 8 grams of diethylenetriamine (Aldrich) was then added to the solution. The resulting solution was a solution of the liquid superabsorbent precursor.

In Sample 2, the sodium hydroxide was increased to 56.8 g, while all the other conditions were kept the same as Sample 1. In Sample 3, the sodium hydroxide was increased to 59.4 g, while all the other conditions were kept the same as Sample 1. In Sample 4, the sodium hydroxide was increased to 61.9 g, while all the other conditions were kept the same as Sample 1.

TABLE 1

| | | Polymer | | |
|---|---|---|---|---|
| Sample | | Solid | ISOBAM ™ | Viscosity |
| No. | ISOBAM ™ Type | Content (%) | Hydrolysis (%) | (cps) |
| 1 | ISOBAM-18 ™ | 20 | 52.5 | 9500 |
| 2 | ISOBAM-18 ™ | 20 | 55.0 | |
| 3 | ISOBAM-18 ™ | 20 | 57.5 | 11600 |
| 4 | ISOBAM-18 ™ | 20 | 60.0 | 12400 |

Viscosity of the liquid superabsorbent precursor solutions was determined on a Rheometric Model DSR 200 stress rheometer using two 25 mm diameter, circular, parallel plates. The instrument was produced by Rheometric Scientific, Inc., Piscataway, N.J. The apparent viscosity of the superabsorbent precursor solution was measured during a stress sweep in a range of about 0.5 Pa to about 1000 Pa. Viscosity was also measured during a frequency sweep ranging from 0.1 to 100 rad/s, at a temperature of 22° C. The apparent viscosity at a shear rate of 0.1 1/s was reported.

The solution prepared in Sample 1 had a viscosity of 9500 centipoise (cps) at a shear rate of 0.1 1/s and a temperature of 22° C. The viscosity of the solution prepared in Sample 3 was 11,600 cps at a shear rate of 0.1 1/s and a temperature of 22° C. The solution made in Sample 4 was 12,400 cps at a shear rate of 0.1 1/s and a temperature of 22° C. All four solutions can be used for printing onto the substrate.

The printing resolution of the superabsorbent solutions is related to the solution viscosity. Generally, when the viscosity of the superabsorbent solutions is about 3000 to 4000 cps and below, the resolution of the printed regions is less than desired.

### Example 3

By replacing one of the high-loft bonded carded web layers on the elastomeric high-loft bonded carded web with, for example, a meltspun web, and placing the superabsorbent on just the non-meltspun side, structures like headbands or wristbands have been made by heat sealing the meltspun material together forming a tube with the superabsorbent side out. Then the tube was turned inside out leaving the meltspun web without superabsorbent on it on the outside. This tube was then heat sealed together at the ends forming the sweatband. Of course, other fastening means like hook and loop (VELCRO) could be used to make the article adjustable. Since the material is elastic, other fastening means are not necessary, but are optional. To further improve the intake and distribution properties small amounts of wood pulp fluff can also be placed inside the sweatband to increase capillarity and thus help draw the fluid to the superabsorbent which then locks up the fluid.

### Test Methods

### Stirbar Retention Test

This test is a measure of superabsorbent attachment to a web when the superabsorbent has been fully swollen. This test was designed to simulate the worst conditions that a superabsorbent nonwoven composite would encounter inside of a swim pant.

The procedure for carrying out the stirbar retention test is as follows:

1. From the material being tested, cut out a 3 centimeter (cm) by 3 cm sample of known or determined substrate basis weight or of known initial SAP concentration.

2. Place the sample in an oven at 105 degrees Celsius for one hour to obtain an initial dry weight.

3. Measure and record the sample initial weight.

4. Soak the sample in an excess of 0.9 weight percent saline for 30 minutes.

5. Place the sample in a 250 ml beaker filled with 200 ml of tap water.

6. Stir at 400 rpm using a 9 millimeter (mm) by 37 mm magnetic, TEFLON-coated stirbar for 5 minutes.

7. Place the sample in oven overnight (16 hours) at 80 degrees Celsius.

8. Measure and record the sample final dry weight.

9. Calculate the percent retention using the following expression:

$$\% SAP retained = 100 \times ( 1 - \frac{initial weight - final weight}{initial weight \times initial SAP concentration } )$$

where “initial SAP concentration,” if known, must be corrected to be on a 1 hour, 105 degree Celsius dry basis, or if the substrate baseis weight is known or can be determined is equal to:

1−(substrate basis weight in gsm×0.0009/initial weight).

### Forced Failure Test

A forced failure test is a means of evaluating the performance of absorbent article prototypes in a controlled manner using live test subjects. Subjects that meet the anatomical requirements of the article being tested are recruited. Test liquid, typically 0.9% saline solution, is introduced into the test product via specially designed belts that contain rubber tubing. The belts are typically positioned on the subject in a manner such that the end of the tubing, where the test liquid comes out, is in the approximate position of the subject's natural anatomical opening, i.e. the gender-specific differences are reflected in the position of the tube's end. The belt is connected to a peristaltic pump that is configured to deliver a desired liquid insult amount at a delivery rate appropriate for the age of the subject. The test article is placed on the subject after the insult delivery system is in place and the testing begins. Liquid insults are delivered to the product, with the subject in a designated insult position, i.e. sitting, standing, prone, supine, etc. Liquid insults are delivered at desired intervals until the product fails via liquid leakage out of the test article. At that time, the article is removed from the subject and the wet weight of the article is recorded to determine the amount of liquid contained inside the article at the point of failure. An average loading at failure is then calculated for each code tested. The following protocol describes a forced failure methodology used to evaluate disposable swimwear prototypes:

14 subjects are recruited in the weight range of 19-34 pounds.

Each subject tests 2 articles per code.

The dry weight of the pant is recorded.

The liquid delivery belt is placed on the subject.

The test article is placed on the subject.

A pair of sweat pants is placed on the subject to help detect liquid leaks.

Liquid is delivered to the subject while in the sitting position.

Liquid insults are delivered via a peristaltic pump at a rate of 15 ml per second.

Liquid insults are spaced by 10 minutes of play time.

The loading sequence involves a first insult size of 60 ml. All subsequent insults are 20 ml until the product fails.

Product failure is defined as a liquid leakage spot on the test sweat pants of about 2.4 centimeters or larger.

After product failure, the test article is removed from the subject.

The wet weight of the test article is measured and recorded.

The load at failure is calculated with the following formula:

Load at failure=wet weight−dry weight

The average load at failure for each code is calculated.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. An absorbent article, comprising:
an absorbent material including superabsorbent polymer non-adhesively attached to a nonwoven in an isolated edge portion of the article, the material having a superabsorbent retention of at least 50% according to a stirbar retention test.

2. The absorbent article of claim 1, wherein the isolated edge portion comprises a pair of containment flaps.

3. The absorbent article of claim 1, wherein the isolated edge portion comprises a waistband area.

4. The absorbent article of claim 1, wherein the isolated edge portion comprises a leg opening area.

5. The absorbent article of claim 1, wherein the isolated edge portion comprises refastenable tabs.

6. The absorbent article of claim 1, wherein the isolated edge portion comprises a pair of side panels.

7. The absorbent article of claim 1, wherein the absorbent article further comprises an absorbent material including superabsorbent polymer non-adhesively attached to a nonwoven in a central portion of the article.

8. The absorbent article of claim 7, wherein the absorbent material in the isolated edge portion of the article at least partially surrounds the absorbent material in the central portion of the article.

9. The absorbent article of claim 1, wherein the absorbent material has a superabsorbent retention of at least 60% according to the stirbar retention test.

10. The absorbent article of claim 1, wherein the absorbent material has a superabsorbent retention of at least 70% according to the stirbar retention test.

11. The absorbent article of claim 1, wherein the absorbent material has a superabsorbent retention of at least 80% according to the stirbar retention test.

12. The absorbent article of claim 1, wherein the superabsorbent polymer is restrained by the nonwoven.

13. The absorbent article of claim 1, wherein the absorbent material is constrained from swelling in thickness by more than about 150% more in tap water than in 0.9 weight percent sodium chloride solution.

14. The absorbent article of claim 1, wherein the absorbent material is constrained from swelling in thickness by more than about 100% more in tap water than in 0.9 weight percent sodium chloride solution.

15. The absorbent article of claim 1, wherein the absorbent material is constrained from swelling in thickness by more than about 75% more in tap water than in 0.9 weight percent sodium chloride solution.

16. The absorbent article of claim 1, wherein the absorbent material is constrained from swelling in thickness by more than about 50% more in tap water than in 0.9 weight percent sodium chloride solution.

17. The absorbent article of claim 1, wherein the superabsorbent polymer is applied to the nonwoven through a printing process.

18. The absorbent article of claim 1, wherein the superabsorbent polymer is applied to the nonwoven through a spraying process.

19. The absorbent article of claim 1, wherein the superabsorbent polymer is applied to the nonwoven through a coating process.

20. The absorbent article of claim 1, wherein the superabsorbent polymer is applied to the nonwoven through a saturation process.

21. The absorbent article of claim 1, wherein the superabsorbent polymer is in a solution comprising a crosslinker, and the superabsorbent polymer is attached to the nonwoven through crosslinking.

22. The absorbent article of claim 1, wherein the superabsorbent polymer is applied to the nonwoven by spraying a superabsorbent monomer solution onto the nonwoven, exposing the superabsorbent monomer solution to radiation, and heating the irradiated solution to remove moisture.

23. The absorbent article of claim 1, wherein two superabsorbent precursor solutions, each containing one part of a redox pair, are combined to initiate polymerization and are applied to the nonwoven before polymerization is completed to form superabsorbent firmly attached to the nonwoven.

24. The absorbent article of claim 1, wherein the nonwoven is coated with the superabsorbent polymer on one side, and the nonwoven is cut into pledgets and then combined with other absorbent materials to form a layered absorbent structure.

25. The absorbent article of claim 1, wherein the superabsorbent polymer is in a solution at a concentration of between about 5% and about 30% by weight.

26. The absorbent article of claim 1, wherein the superabsorbent polymer is in a solution at a concentration of between about 10% and about 25% by weight.

27. The absorbent article of claim 1, wherein the superabsorbent polymer is in a solution at a concentration of between about 15% and about 22% by weight.

28. The absorbent article of claim 1, wherein the superabsorbent polymer is in a solution with a viscosity of between about 3,000 and about 20,000 centipoise at a shear rate of 0.1 1/s and a temperature of 22 degrees Celsius.

29. The absorbent article of claim 1, wherein the superabsorbent polymer is in a solution with a viscosity of between about 5,000 and about 15,000 centipoise at a shear rate of 0.1 1/s and a temperature of 22 degrees Celsius.

30. The absorbent article of claim 1, wherein the superabsorbent polymer is in a solution with a viscosity of between about 7,000 and about 13,000 centipoise at a shear rate of 0.1 1/s and a temperature of 22 degrees Celsius.

31. The absorbent article of claim 1, wherein the nonwoven comprises at least one of a group consisting of a meltspun web, a meltblown web, coform, an elastomeric surge, a meltspun laminate, an elastomeric meltspun laminate, a bonded carded web, a high loft bonded carded web, and an elastomeric high loft bonded carded web.

32. The absorbent article of claim 1, wherein the absorbent material has a centrifuge retention capacity of between about 4 grams per gram and about 30 grams per gram.

33. The absorbent article of claim 1, wherein the absorbent material has a centrifuge retention capacity of between about 5 grams per gram and about 25 grams per gram.

34. The absorbent article of claim 1, wherein the absorbent material has a centrifuge retention capacity of between about 6 grams per gram and about 20 grams per gram.

35. The absorbent article of claim 1, wherein the absorbent article is one selected from a group consisting of personal care garments, medical garments, athletic garments, and workwear garments.

36. The absorbent article of claim 1, wherein the absorbent article is one selected from a group consisting of diapers, training pants, swim wear, absorbent underpants, adult incontinence products, feminine hygiene products, nursing pads, underarm pads, wipes, breathable-when-dry outer absorbent product covers, protective medical gowns, surgical medical gowns, bandages, caps, gloves, drapes, face masks, laboratory coats, coveralls, athletic socks, pants, supporters, bras, shirts, hard-hat liners, helmet liners, sweatbands, table cloths, and paper tissue.

37. The absorbent article of claim 1, further comprising a wetness indicator including the absorbent material.

38. An absorbent article, comprising:
an absorbent material including superabsorbent polymer non-adhesively attached to a nonwoven in a discreet zone of the nonwoven, the nonwoven folded over the discreet zone such that the discreet zone is encompassed within the folded nonwoven, the material having a superabsorbent retention of at least 50% according to a stirbar retention test.

39. The absorbent article of claim 38, wherein the nonwoven is e-folded around the discreet zone.

40. The absorbent article of claim 38, wherein the nonwoven is c-folded around the discreet zone.

41. The absorbent article of claim 38, wherein the nonwoven is z-folded around the discreet zone.

42. An absorbent article, comprising:
one of a group consisting of medical garments, athletic garments, and workwear garments, including superabsorbent polymer non-adhesively attached to a nonwoven and having a superabsorbent retention of at least 50% according to a stirbar retention test.

43. The absorbent article of claim 42, wherein the superabsorbent polymer non-adhesively attached to the nonwoven has a superabsorbent retention of at least 60% according to the stirbar retention test.

44. The absorbent article of claim 42, wherein the superabsorbent polymer non-adhesively attached to the nonwoven has a superabsorbent retention of at least 70% according to the stirbar retention test.

45. The absorbent article of claim 42, wherein the superabsorbent polymer non-adhesively attached to the nonwoven has a superabsorbent retention of at least 80% according to the stirbar retention test.

46. The absorbent article of claim 42, wherein the superabsorbent polymer non-adhesively attached to the nonwoven has a centrifuge retention capacity of about 4 grams per gram to about 30 grams per gram.

47. The absorbent article of claim 42, wherein the superabsorbent polymer non-adhesively attached to the nonwoven has a centrifuge retention capacity of about 5 grams per gram to about 25 grams per gram.

48. The absorbent article of claim 42, wherein the nonwoven comprises at least one of a group consisting of a meltspun web, a meltblown web, coform, an elastomeric surge, a meltspun laminate, an elastomeric meltspun laminate, a bonded carded web, a high loft bonded carded web, and an elastomeric high loft bonded carded web.

49. An absorbent swim wear garment, comprising:
a chassis defining a waist opening and first and second leg openings, the chassis including a body side liner, an outer cover, and an absorbent layer between the body side liner and the outer cover, the absorbent layer including superabsorbent polymer non-adhesively attached to a nonwoven and having a superabsorbent retention of at least 50% according to a stirbar retention test.

50. The swim wear garment of claim 49, wherein the absorbent layer is in an isolated edge portion of the garment.

51. The swim wear garment of claim 49, wherein the absorbent layer is in a central portion of the garment.

52. The swim wear garment of claim 49, wherein the absorbent layer has a superabsorbent retention of at least 60% according to the stirbar retention test.

53. The swim wear garment of claim 49, wherein the absorbent layer has a superabsorbent retention of at least 70% according to the stirbar retention test.

54. The swim wear garment of claim 49, wherein the absorbent layer has a superabsorbent retention of at least 80% according to the stirbar retention test.

55. The swim wear garment of claim 49, wherein the absorbent layer has a centrifuge retention capacity of about 4 grams per gram to about 30 grams per gram.

56. The swim wear garment of claim 49, wherein the absorbent layer has a centrifuge retention capacity of about 5 grams per gram to about 25 grams per gram.

57. The swimwear garment of claim 49, wherein the superabsorbent polymer is applied to the nonwoven through a printing process.

58. The swimwear garment of claim 49, wherein the superabsorbent polymer is applied to the nonwoven through a spraying process.

59. The swimwear garment of claim 49, wherein the superabsorbent polymer is in a solution comprising a crosslinker, and the superabsorbent polymer is attached to the nonwoven through crosslinking.

60. The swimwear garment of claim 49, wherein the superabsorbent polymer is applied to the nonwoven by spraying a superabsorbent monomer solution onto the nonwoven, exposing the superabsorbent monomer solution to radiation, and heating the irradiated solution to remove moisture.

61. The swimwear garment of claim 49, wherein the superabsorbent polymer is applied to the nonwoven as a monomer solution with a redox initiator system such that polymerization and cross-linking is completed on the nonwoven.

62. The swimwear garment of claim 49, wherein the nonwoven is coated with the superabsorbent polymer on one side, and the nonwoven is cut into pledgets and then combined with other absorbent materials to form a layered absorbent structure.

63. The swimwear garment of claim 49, wherein the nonwoven comprises at least one of a group consisting of a meltspun web, a meltblown web, coform, an elastomeric surge, a meltspun laminate, an elastomeric meltspun laminate, a bonded carded web, a high loft bonded carded web, and an elastomeric high loft bonded carded web.
